(19) United States
(12) Patent Application Publication
(10) Pub. No.: US 2015/0253402 A1
(43) Pub. Date: Sep. 10, 2015

(54) METABOLITE DETECTION SYSTEM AND OPERATION THEREOF

(71) Applicant: KONINKLIJKE PHILIPS N.V., EINDHOVEN (NL)

(72) Inventor: Baolian Yang, Highland Heights, OH (US)

(73) Assignee: KONINKLIJKE PHILIPS N.V., EINDHOVEN (NL)

(21) Appl. No.: 14/429,126

(22) PCT Filed: Sep. 9, 2013
(86) PCT No.: PCT/IB2013/058397

§ 371 Date: Mar. 18, 2015

Related U.S. Application Data
(60) Provisional application No. 61/703,307, filed on Sep. 20, 2012.

(51) Int. Cl. G01R 33/465; A61B 5/00; A61B 5/055

(52) U.S. Cl. CPC G01R 33/465; A61B 5/055; A61B 5/4058

(57) ABSTRACT

A magnetic resonance (MR) system for detecting concentrations of one or more metabolites in a volume of interest (VOI), the system including at least one controller which: may apply to the VOI a multiple quantum filter (MQF) Point Resolved Spetroscopy (PRESS) sequence comprising first and second 90° RF pulses, a third 90° RF pulse, first and second 180° adiabatic pulses, and a composite dual-band delay alternating with nutation for tailored excitation (DANTE) pulse train having a plurality of N block pulses (N being an integer), the DANTE pulse train situated in time between the first and second 90° RF pulses, the first and second 180° adiabatic pulses situated in time after third 90° RF pulse; detect MR Free Induced Decay (FID) signal emitted from the VOI; and/or reconstruct the detected MR FID signal to obtain metabolite spectrum information.

### PRIORITY CLAIM

This application claims priority from U.S. Provisional Application No. 61/703,307, filed Sep. 20, 2012 the contents of which are incorporated herein by reference.

### FIELD

Exemplary embodiments of the present invention can provide medical diagnostic imaging including a magnetic resonance (MR) system for detecting one or more metabolites such as γ-Aminobutyrate acid (GABA) and, more particularly, to an automated in vivo metabolite detection system and a method of operation thereof.

### BACKGROUND INFORMATION

Magnetic resonance spectroscopy (MRS) is a form of magnetic resonance imaging (MRI) and can be is used to measure concentrations of metabolites in test subjects (e.g., a mammal, etc.) in vivo. One of these metabolites is known as γ-Aminobutyric acid (GABA) and is often referred to as the chief inhibitory neurotransmitter in the mammalian (e.g., human) central nervous system. Unfortunately, GABA is often implicated in several neuropsychiatric disorders in humans such as Parkinsons disease. Typically, GABA concentration in a normal human cranium (e.g., a human brain) is approximately 1 mM which is a concentration that is near limit of detectability of conventional MRS systems. Further, when using MRS to detect a GABA concentration in vivo in a human cranium, a GABA signal spectra is often overlapped with a signal spectra of other high-concentration metabolites present in the cranium of the test subject such as Creatine (Cr) and Macromolecule (MM). Accordingly, it is difficult, if not impossible, to accurately detect GABA concentrations in vivo in mammals (e.g., see, Nicolass A. J. Puts, Richard Edden, Progress in Nuclear Magnetic Resonance Spectroscopy (2012), 60:29-41, the contents of which are incorporated herein by reference) using conventional MRS methods.

With regard to MRS methods used to measure concentrations of metabolites such as GABA in test subjects, these methods may include: (a) a J-difference (e.g., J-difference technique) technique; (b) a multiple quantum filter (MQF) technique; and (c) a two-dimensional MRS (2D MRS) method. The J-difference technique is described by Kevin W. Waddell, et al., MRI, 2007, 25: 1032-1038 (Waddell), the contents of which are incorporated herein by reference; and the MQF technique is described by Changho Choi, et al., MRM, 2005, 54:272-279, (Choi, et al.); and Helen Geen, et al., JMR, 1989, 81:646-652 (Geen, et al.), the contents of each of which are incorporated herein by reference.

Generally, the J-difference technique is used in conjunction with three Tesla (3T) MRI systems and is popular due to its greater sensitivity when compared to MQF and 2D MRS methods. However, even with its greater sensitivity, results of the J-difference technique are often unreliable and/or inaccurate due to background signal contamination of a signal spectra due to for example Cr and MM concentrations present in a volume of interest (VOI) being sampled such as a human cranium. Fortunately, with the advent of 3T systems using phase array coils (PACs) (e.g., up to 32 channels) an improvement in SNR may be realized during MR clinical scans.

### SUMMARY

The system, device(s), method, user interface, computer program, etc., (hereinafter each of which will be referred to as system unless the context indicates otherwise) described herein addresses problems in prior art systems. In accordance with embodiments of the present system, a system for the non-invasive detection of GABA in vivo in a cranium (e.g., in a brain) of a test subject can be provided. Further, the present system may provide robust detection, low background signal contamination, and a high signal-to-noise ratio (SNR). Further, it is envisioned that embodiments of the present system may provide a flexible dual-band pulse design MRI system for accurately detecting metabolites such as GABA in a test subject (e.g., a mammal, etc.) in vivo using non-invasive techniques.

In accordance with an aspect of the present system, a magnetic resonance (MR) system for detecting concentrations of one or more metabolites in a volume of interest (VOI) of a test subject can be provided. The system can include at least one controller which: may apply to the VOI a multiple quantum filter (MQF) Point Resolved Spetroscopy (PRESS) sequence comprising first and second 90° RF pulses, a third narrow-band 90° RF pulse, first and second 180° adiabatic pulses, and a composite dual-band delay alternating with nutation for tailored excitation (DANTE) pulse train having a plurality of N block pulses (N being an integer), the DANTE pulse train situated in time between the first and second 90° RF pulses, the first and second 180° adiabatic pulses situated in time after the third 90° RF pulse; detect MR echo information emitted from the VOI; and/or reconstruct the detected MR echo information to obtain metabolite spectrum information.

It is further envisioned that the controller may control application of the first and second 90° RF pulses which may each have a non-symmetric Spredrex-type waveform, the third 90° excitation RF pulse (e.g., a third narrow band 90° excitation RF pulse) may have a SINC center type waveform. Further, the first and second 180° adiabatic pulses may each have an oit_{—}800_{—}6500 pulse-type waveform (OIT: Offset Independent Trapezoid adiabatic inversion pulse, has RF B₁≧800 Hz, and an excitation bandwidth=6500 Hz). It is further envisioned that the DANTE pulse train may be constructed in accordance with following parameters: PD_tot=1.0/BW; τ=0.5/BW_side; N=PD_tot/τ; Flip=2*Flip_tot/N; θ=−4*τ*BW_diff*180°; Phase_even=(N/2−1) θ, (N/2−2) θ, . . . θ, 0°; and/or Odd_Pulse phase=0°, where, BW refers to an excitation band of the DANTE pulse train in Hz, PD_tot is a pulse total duration, T is a block pulse interval, BW_side refers to a sideband of a DANTE pulse, N is an integer corresponding to a total number of block pulses, Flip is a flip angle of each block pulse, Flip_tot refers to a flip angle of the pulse, θ is phase-shift step of even pulses, BW_diff refers to the distance of dual bands, Phase_even is a phase of even pulses, and Odd_Pulse phase refers to a phase of each odd pulse. It is further envisioned that a selected metabolite of the one of more metabolites may be γ-Aminobutyric acid (GABA) and that the controller may determine a concentration of the selected metabolite in accordance with the metabolite spectrum information.

In accordance with yet another aspect of the present system, a method of detecting concentrations one or more metabolites in a volume of interest (VOI) of a test subject using magnetic resonance spectroscopy (MRS) can be provided. The method can be performed by a controller of a MRS system and can include one or more acts of: applying to the VOI a multiple quantum filter (MQF) Point Resolved Spetroscopy (PRESS) sequence comprising first and second 90° RF pulses, a third 90° RF pulse, such as a third narrow-band 90° RF pulse, first and second 180° adiabatic pulses, and a composite dual-band delay alternating with nutation for tailored excitation (DANTE) pulse train having a plurality of N block pulses, the DANTE pulse train situated in time between the first and second 90° RF pulses, the first and second 180° adiabatic pulses situated in time after the third 90° RF pulse; detecting MR echo information emitted from the VOI; and reconstructing the detected MR echo information to obtain metabolite spectrum information.

It is further envisioned that the method may further include an act of applying the first and second 90° RF pulses each having a non-symmetric Spredrex-type waveform, the third narrow band 90° excitation RF pulse may have a SINC center type waveform. Moreover, the first and second 180° adiabatic pulses have an oit_{—}800_{—}6500 pulse-type waveform (Offset Independent Trapezoid adiabatic inversion pulse). Further, it is envisioned that the method may include an act of constructing the DANTE pulse train in accordance with following parameters: PD_tot=1.0/BW; τ=0.5/BW_side; N=PD_tot/τ; Flip=2*Flip_tot/N; θ=−4*τ*BW_diff*180°; Phase_even=(N/2−1) θ, (N/2−2) θ, . . . θ, 0°; and/or Odd_Pulse phase=0°. It is further envisioned that the method may further comprising an act of determining a concentration of the selected metabolite in accordance with the metabolite spectrum information, wherein a selected metabolite of the one of more metabolites is γ-Aminobutyric acid (GABA).

In accordance with yet a further aspect of the present system, a computer program stored on a computer readable memory medium can be provided. The computer program can be configured to detect concentrations one or more metabolites in a volume of interest (VOI) of a test subject using magnetic resonance spectroscopy (MRS), the computer program may include a program portion configured to: apply to the VOI a multiple quantum filter (MQF) Point Resolved Spetroscopy (PRESS) sequence comprising first and second 90° RF pulses, a third 90° RF pulse, such as a third narrow-band 90° RF pulse, first and second 180° adiabatic pulses, and a composite dual-band delay alternating with nutation for tailored excitation (DANTE) pulse train having a plurality of N block pulses, the DANTE pulse train situated in time between the first and second 90° RF pulses, the first and second 180° adiabatic pulses situated in time after the third 90° RF pulse; detect MR echo information emitted from the VOI; and reconstruct the detected MR echo information to obtain metabolite spectrum information.

It is further envisioned that the program portion may be configured to form the first and second 90° RF pulses in accordance with a non-symmetric Spredrex-type waveform and the third narrow-band 90° RF pulse in accordance with a SINC center waveform. It is also envisioned that the program portion may be configured to form the first and second 180° adiabatic pulses in accordance with an oit_{—}800_{—}6500 pulse-type waveform. It is further envisioned that the program portion may be configured to construct the DANTE pulse train in accordance with following parameters: PD_tot=1.0/BW; τ=0.5/BW_side; N=PD_tot/τ; Flip=2*Flip_tot/N; θ=−4*τ*BW_diff*180°; Phase_even=(N/2−1) θ, (N/2−2) θ, . . . θ, 0°; and/or Odd_Pulse phase=0°. It is further envisioned that the program portion may be configured to determine a concentration of the selected metabolite in accordance with the metabolite spectrum information, wherein the selected metabolite of the one of more metabolites may be γ-Aminobutyric acid (GABA).

### BRIEF DESCRIPTION OF THE DRAWINGS

The present system is explained in further detail, and by way of example, with reference to the accompanying drawings wherein:

FIG. 1 is a screen shot illustrating results of a band selection of a DANTE pulse train in accordance with embodiments of the present system;

FIG. 2 is a schematic diagram illustrating an MQF PRESS pulse sequence for a single repetition time (TR) in accordance with embodiments of the present system;

FIG. 3 is a graph illustrating spectra of five dynamic scans obtained in accordance with the parameters of Test-1 in accordance with embodiments of the present system;

FIG. 4 is a screen shot illustrating spectra obtained in accordance with Test-2 parameters upon a 1 mM GABA phantom in accordance with embodiments of the present system;

FIG. 5 shows a flow diagram that illustrates a process performed on an MRI system in accordance with embodiments of the present system;

FIG. 6 is a block diagram of an MR imaging system in accordance with embodiments of the present system; and

FIG. 7 shows a portion of a system (e.g., peer, server, etc.) in accordance with an embodiment of the present system.

### DETAILED DESCRIPTION

The following are descriptions of illustrative embodiments that when taken in conjunction with the following drawings will demonstrate the above noted features and advantages, as well as further ones. In the following description, for purposes of explanation rather than limitation, illustrative details are set forth such as architecture, interfaces, techniques, element attributes, etc. However, it will be apparent to those of ordinary skill in the art that other embodiments that depart from these details would still be understood to be within the scope of the appended claims. Moreover, for the purpose of clarity, detailed descriptions of well known devices, circuits, tools, techniques and methods are omitted so as not to obscure the description of the present system. It should be expressly understood that the drawings are included for illustrative purposes and do not represent the scope of the present system. In the accompanying drawings, like reference numbers in different drawings may designate similar elements. The technique includes generating:
(1) A dual-band DENTA pulse (DBDP) train (sequence) that may be flexiblely applied wherein the DBDP pulse train may for example be generated on a scanner in real time according to an adjustable bandwidth for each band. In this way, the distance between two bands may be adjusted to enable the sequence to be easily adjusted to detect different metabolites such as GABA, one or more other macromolecules, a human scan, a phantom scan, etc.; and
(2) a MQF Point Resolved Spetroscopy (PRESS) sequence.

With regard to the DBDP train, this pulse train includes a dual-band excitation radio frequency (RF) pulse train generated in real time and includes a dual-band delay alternating with nutation for tailored excitation (DANTE) pulse train (e.g., a small flip angle block pulse train). The DBDP may be set in accordance with DBDP settings and may be set independently of field strength of a main coil. Thus, the same DBDP train may be used for example for 1.5T, 3T and 7T MRI systems. Moreover, with regard to pulse duration, the pulse duration of the DBDP does not substantially depend upon a distance between two selection bands of the DBDP. Therefore, various reasonable settings may be set for pulse duration. The selection bandwidth of each band of this DBDP pulse may, for example, be substantially in the range of 40 Hz˜150 Hz, and may, for example, have a duration of approximately 25 to 6.7 ms. The term substantially, about, ˜, and other like terms and expressions as utilized herein are definite and should be understood to include ranges, values, etc., that are also beyond the range, value, etc. such as, e.g., +/−10% of range, values and/or otherwise within precision of related instrument settings.

With regard to the MQF PRESS sequence, this sequence is generated so that it preferably has an echo time (TE) for example substantially=68 ms (e.g., 66 ms˜72 ms) with adiabatic refocus pulses which may provide an accurate volume selection and a reduced chemical shift artifact. The MQF PRESS sequence with settings AS illustratively described herein may be used to detect concentrations of GABA. In yet other embodiments, it is envisioned that the MQF PRESS sequence may be adjusted to detect a concentration of various selected metabolites such as MM, etc. This may be performed by adjusting the selection bandwidth of each band of DBDP pulse, the distance between two excitation bands of this pulse, and the center frequency of the third 90° RF pulse.

Design of the Flexible Dual Band Denta Pulse (DBDP) Train

In accordance with embodiments of the present system, a DBDP train includes a dual-band excitation RF pulse train which may be generated by any suitable MRI scanner such as, e.g., a 3.0T Philips™ Achieva™ MRI scanner. The DBDP RF pulse train is an element of an MQF PRESS of the present system and improves the efficiency of multiple quantum coherence to enhance a signal spectra indicative of a concentration of one or more selected metabolites (e.g., GABA in the present embodiment) and suppress signals indicative of concentrations of non-selected metabolites (e.g., Cr in the present example) whose spectra may be overlapped with a spectra of the selected metabolite. For the sake of clarity, the signals indicative of a detected concentration of a metabolite (e.g., GABA) will be commonly referred to using the name of the metabolite (e.g., a GABA signal).

In accordance with one or more embodiments of the present system, a controller of an MRI scanner may generate the DBDP train having settings in accordance with at least part of the input parameters set forth in Table 1 below. This DBDP train may be referred to as a dual-band DANTE pulse.

TABLE 1
MRI SCANNER SETTINGS FOR DBDP SEQUENCE

Pulse total duration (PD_tot) = 1.0/BW
Block pulse interval (τ) = 0.5/BW_side
Number of block pulses (N) = PD_tot/τ
Flip angle of each block pulse (Flip) = 2 * Flip_tot/N
Phase of each odd pulse = 0°
Phase-shift step of even pulse (θ) = −4 * τ * BW_diff * 180°
Phase of even pulse = (N/2 − 1) θ, (N/2 − 2) θ, . . . θ, 0

where, BW refers to an excitation bandwidth of an individual band in Hz; BW_diff refers to the distance between the two excitation bands; BW_side refers to a sideband of a DANTE pulse; pdc refers to a block pulse duty cycle; Flip_tot refers to the flip angle of the DBDP pulse for the spins in each excitation band; N is a number such as an integer number of block pulses used in the DBDP train; Flip refers to a flip angle of each block pulse; θ refers to a phase-shift step of even pulses in the DBDP train (sequence). PD_tot is the total duration of DBDP pulse and may be substantially in the range of 25˜6.7 ms. For example, in accordance with one or more embodiments of the present system, to generate the pulse, the following parameters may be set first according to the metabolite to be detected: BW (e.g., substantially in a range of 40 Hz-150 Hz) BW_diff (e.g., substantially in a range of 50-300 Hz), BW_side (e.g., substantially in a range of 1000 Hz-2000 Hz), Flip_tot (e.g., substantially in a range of 90 °˜180°), pdc (e.g., substantially in a range of 10%˜20%), then other parameters of the pulse may be calculated according the relationship listed in Table 1.

These parameters may be set by a user and/or system and/or may be stored in a memory of the system for later use. Accordingly, when desired, the system may obtain the parameters such as those set forth in Table 1 from a user and/or from a memory of the system and determine values for each corresponding parameter settings in accordance with embodiments of the present system. The values for one or more corresponding parameters may be set by a user and/or the system and may be stored in association with the parameters in a memory of the system for later use. The values and/or parameters may be further associated with a given user, patient, and/or test type (e.g., GABA test, Glutamine Test, etc.). Thereafter, when one of the associated user, patient, and/or test type (e.g., a GABA test in the present example) is selected by the system and/or user such as via a menu item selection on a user interface, such as a graphical user interface (GUI), a direct input (e.g., “RUN GABA TEST”) of the user, or is otherwise selected, the system may obtain the corresponding parameters and/or settings and generate the DBDP train accordingly.

Exemplary Test Results

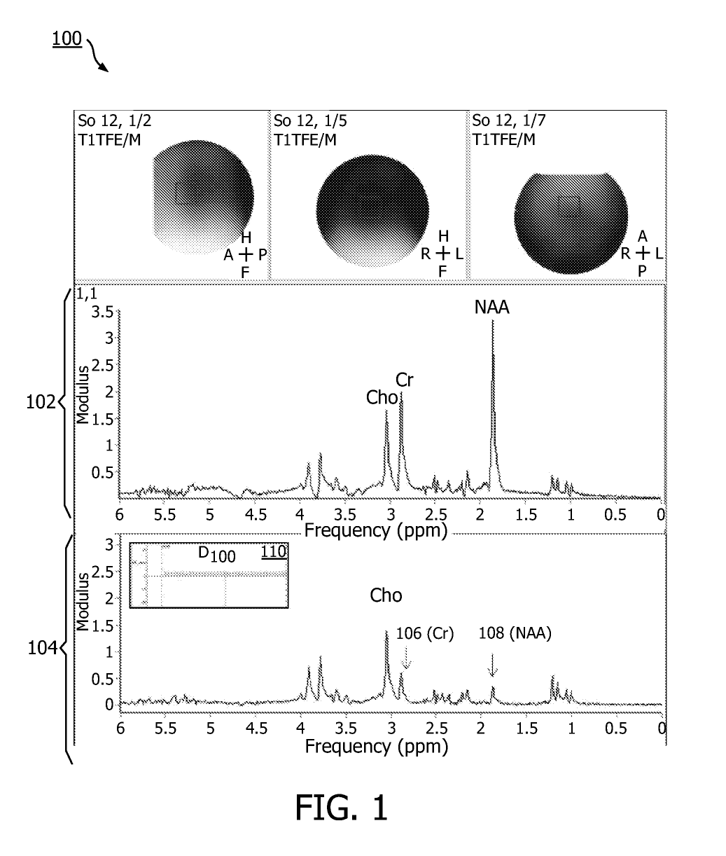

FIG. 1 is a screen shot 100 illustrating results of a DBDP pulse D₁₀₀ in accordance with embodiments of the present system. To verify the dual band selection of the DBDP train generated in accordance with methods of the present system such as those illustrated above with regard to Table 1 and the corresponding text, a flip angle (Flip_tot) of 100° DBDP pulse which targets on Cr (−230 Hz from water signal at 3T) and N-acetyl aspartate (NAA) (−128 Hz from the Cr signal) signals of a brain phantom (e.g., a GE™ Braino™ phantom) is used as a saturation pulse in this scan on a Philips™ Achieva™ 3.0T TX system including an eight-channel head coil. In accordance with an illustrative embodiment as shown, D₁₀₀ includes 40 block pulses (e.g., N=40) and the duration is 19.7 ms which provides a 50 Hz excitation bandwidth for each individual band. Settings of values of parameters for this test are shown in Table 2 below.

TABLE 2
MRI SCANNER SETTINGS AND VALUES FOR DBDP TRAIN

| Parameter | Value |
|---|---|
| Pulse total duration (PD_tot) = 1.0/BW | PD_tot = 19.7 msec |
| Block pulse interval (τ) = 0.5/BW_side | T = 0.5 ms |
| Number of block pulses (N) = PD_tot/τ | N = 40 |
| Flip angle of each block pulse (Flip) = 2 * | 5° |
| Flip_tot/N | |
| Phase of each odd pulse = 0° | |
| Phase-shift step of even pulse (θ) = −4 * | Θ = 46° |
| τ * BW_diff * 180° | |
| Phase of even pulse = (N/2 − 1) θ, | |
| (N/2 − 2) θ, . . . θ, 0 | |

Referring to line graphs 102 and 104 of FIG. 1, graph 102 illustrates a spectra (e.g., indicating magnitude of concentration) obtained using a PRESS sequence plus water suppression (e.g., TE/TR=50/2000 ms); and graph 104 illustrates a spectra obtained using an additional DBDP pulse D₁₀₀ as pre-saturation pulse in accordance with embodiments of the present system to verify the dual band excitation of DBDP pulse. Insert 110 illustrates the D₁₀₀ pulse train of the DBDP train with the parameters listed in Table 2.

A comparison of the spectra of graphs 102 and 104 shows suppression of peaks of Cr and NAA (as illustrated by arrows 106 and 108, respectively) in the spectrum of the graph 104. This suppression in accordance with one or more embodiments of the present system is due to use of the dual band excitation of D₁₀₀ pulse. More particularly, peaks of NAA and Cr are reduced significantly in the spectrum of the graph 104 and are only 11% and 28%, respectively, of their respective peaks in the spectrum of the graph 102. Further, Cho (0.1 ppm from Cr), Glx (˜2.5 ppm), and lactate (˜1.3 ppm) peaks in the spectra of graph 104 remain substantially constant and are about 88% of their levels in the spectra of graph 102.

This unique dual-band selection allows an MQF PRESS sequence in accordance with the present system to suppress the undesirable signals from other metabolites and detect the signal only from the metabolite of interest such as GABA.

Design of the Mqf Press Pulse Sequence

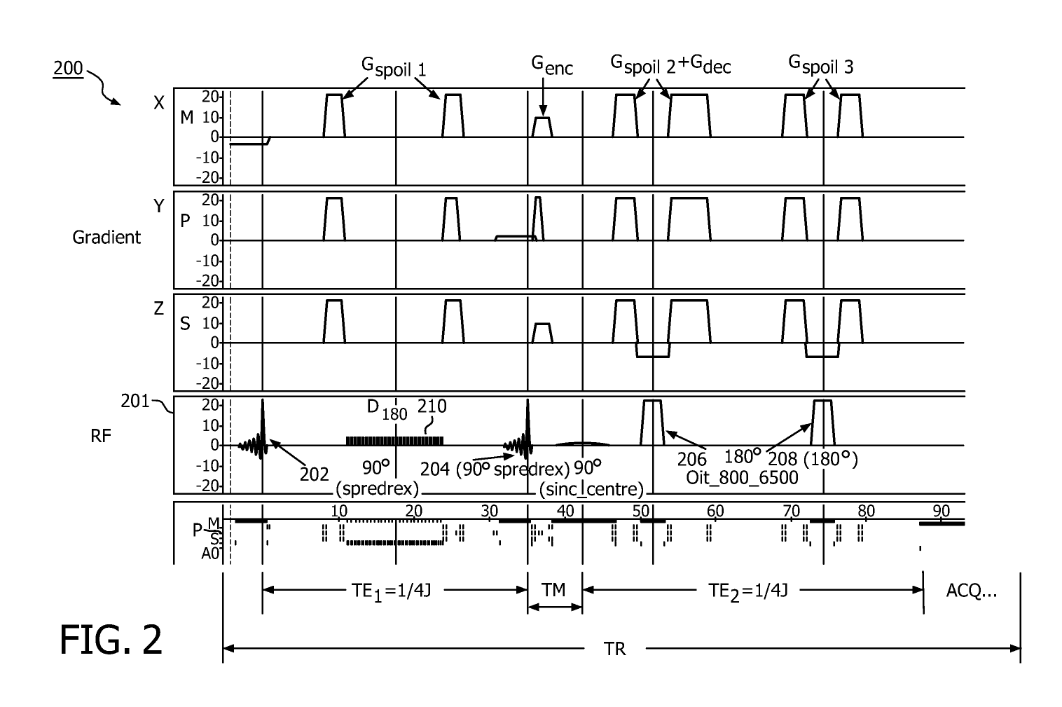

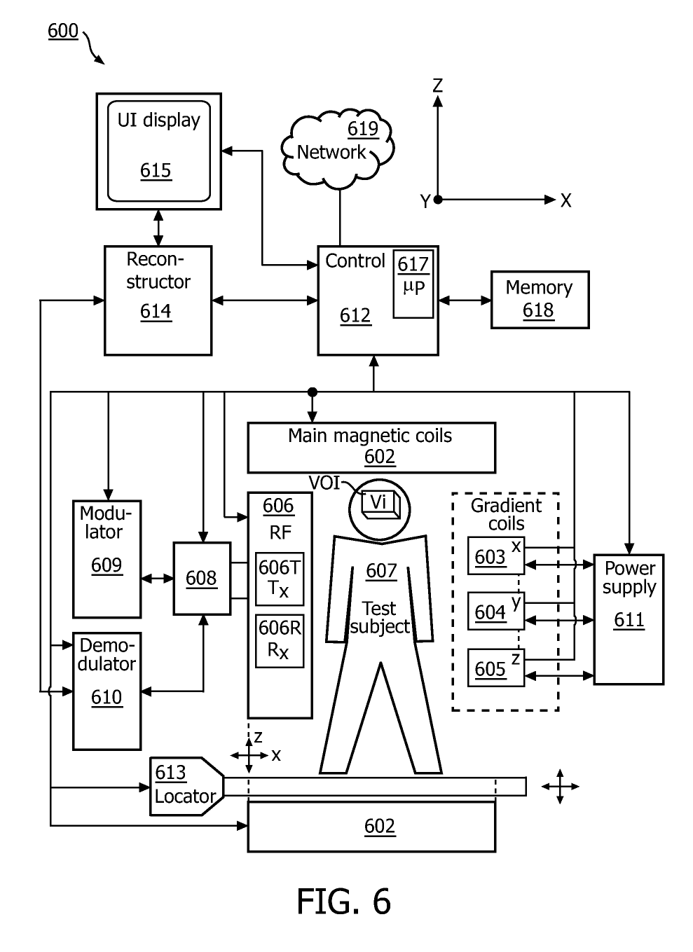

FIG. 2 is a schematic diagram 200 illustrating an MQF PRESS pulse sequence for a single repetition time (TR) to acquire a localized FID (Free Induced Decay) signal from a single voxle in accordance with embodiments of the present system. The MQF PRESS pulse sequence is compatible with various MRI systems such as a 3.0T Philips™ Achieva™ system as illustrated in FIG. 6 below or other suitable MRI systems or parts thereof. During each repetition time (TR), RF energy is exchanged between the imaging system and the test subject. An RF output sequence 201 includes pulses as will be described below. At specific times during each repetition time (TR), material (e.g., tissue of a human test subject, contents of a phantom test subject, etc.) is stimulated to emit RF signals (e.g., echo), which are sensed by sensors of the system. The FID signals are analyzed or otherwise processed to reconstruct a spectrum of detected concentrations of at least one selected metabolite (such as GABA in the present example). The Analysis process may include Fast Fourier Transfer (FFT), and data display.

Referring to FIG. 2, with regard to the RF output sequence 201, this sequence includes first and second 90° RF pulses 202 and 204, respectively, third 90° RF pulse 212 and first and second 180° adiabatic pulses 206, and 208, respectively. In accordance with one or more embodiments of the present system, the pulses 202 and 204 may be utilized respectively for x, and y direction selection, pulses 206 and 208 may be utilized respectively for z direction selection, and the third 90° pulse may be a non-selective SINC pulse which excites GABA H3 signal at 1.89 ppm.

With regard to duration and bandwidth, the first and second 90° RF pulses, 202 and 204, respectively, illustratively have a duration of substantially 4.2 ms and a bandwidth of substantially 3.8 kHz; and the first and second 180° adiabatic pulses 206 and 208, respectively, illustratively have a duration of substantially 3.1 ms and a bandwidth of substantially 8.1 kHz. The third 90° pulse may be substantially a 11.3 ms SINC pulse with a bandwidth of substantially 120 Hz. However, other durations and/or bandwidths are also envisioned.

The 90° RF pulses such as the first and second 90° RF pulses 202 and 204, respectively, have an asymmetric SINC waveform such as an asymmetric waveform formed as Philips™ Spredrex™-type pulses to preferably reduce pulse duration. For example, since TE1 is limited to substantially 34 ms=½J, where J (e.g., substantially 7.3 Hz) is the coupling factor between GAGA H3 and H4 spins. Accordingly, the first and second 180° adiabatic pulses 206, and 208, respectively, may have a waveform which is set in accordance with Philips™ oit_{—}800_{—}6500-type pulses (OIT: offset Independent Trapezoid adiabatic inversion pulse) so as to increase a refocus bandwidth. A single band excitation 90° SINC pulse 212 is situated between the second 90° RF pulse 204 and the first 180° pulse 206. In accordance with one or more embodiments of the present system, first and second 90° pulses are used to create double-quantum coherences, and also work as slice selection pulses in X and Y direction, where DBDP pulse D₁₈₀ will selectively refocus GABA H3/H4 only related coherences. In accordance with one or more embodiments of the present system, the third 90° pulse works as a read pulse which change the selected double-quantum coherences to single-quantum coherences since single-quantum coherence may be detectable in a MRI system. In accordance with one or more embodiments of the present system, the first and second OIT 180° pulses work as Z direction selection pulses to finish the volume selection. All other signals except GABA H4 will be de-phased by spoil gradients.

In accordance with embodiments of the present system, the refocus gradients may be utilized to compensate phase distortion during x and y slice section around first and second 90° RF pulses 202 and 204, respectively, as well as decoding gradients (G_{dec}) are merged with spoil gradients 1 and 2 (G_{spoil 1} and G_{spoil 2}, respectively), with decoding gradients having twice the amplitude of encoding gradients (G_{enc}) for double quantum filtering (G_{dec}⁼2*G_{enc}). The gradient strength of G_{spoil 1}, G_{spoil 2} and G_{spoil 3} in accordance with one or more embodiments of the present system may be substantially 31 mT/m, and the duration may be 1.67, 1.90 and 1.90, respectively. The strength of G_{dec} may be substantially 10 mT/m and the duration may be substantially 2 ms, while the strength of G_{dec} may be substantially 20 mT/m_and may have the same duration as G_{dec}.

In accordance with embodiments of the present system, settings of parameters used for GABA detection at 3T may include the following settings or the substantial equivalent: TE₁=35 ms, TM=8.8 ms, TE=TE₁+TE₂=68 ms, TR=2500 ms. D₁₈₀ (210) illustratively is a 16.7 ms long composite DANTE pulse train with 34 block pulses, which provides a 60 Hz excitation bandwidth, with a center frequency targeted at Cr (3.02 ppm) with a second band at GABA H3 signal (1.89 ppm). The third 90° SINC pulse 212 may have a duration of substantially 11.3 ms is on GABA H3 signal with an 120 Hz excitation bandwidth. These settings may be stored in a memory of the system (e.g., in a function library, etc.) for later use. Further, these settings (in whole or in part) may be set by the user and/or system. In accordance with one or more embodiments of the present system J is the coupling factor between GABA H3 and H4 spins, TE1 is the first echo time, and TE2 is the second echo time, where TE=TE1+TE2, TM is the mixing time, and ACQ is data acquisition.

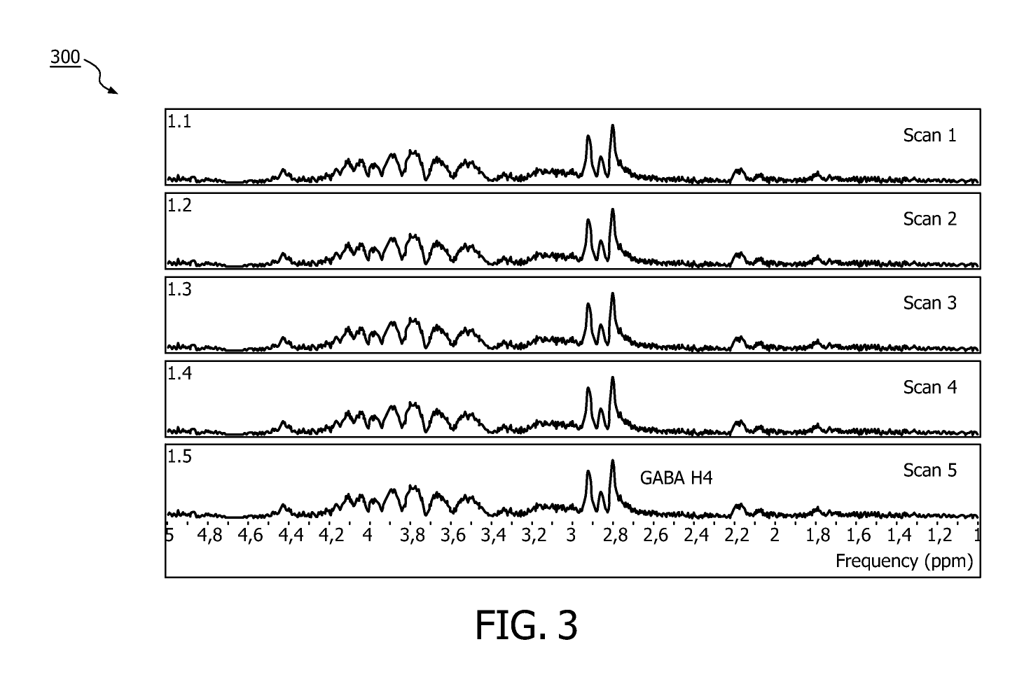

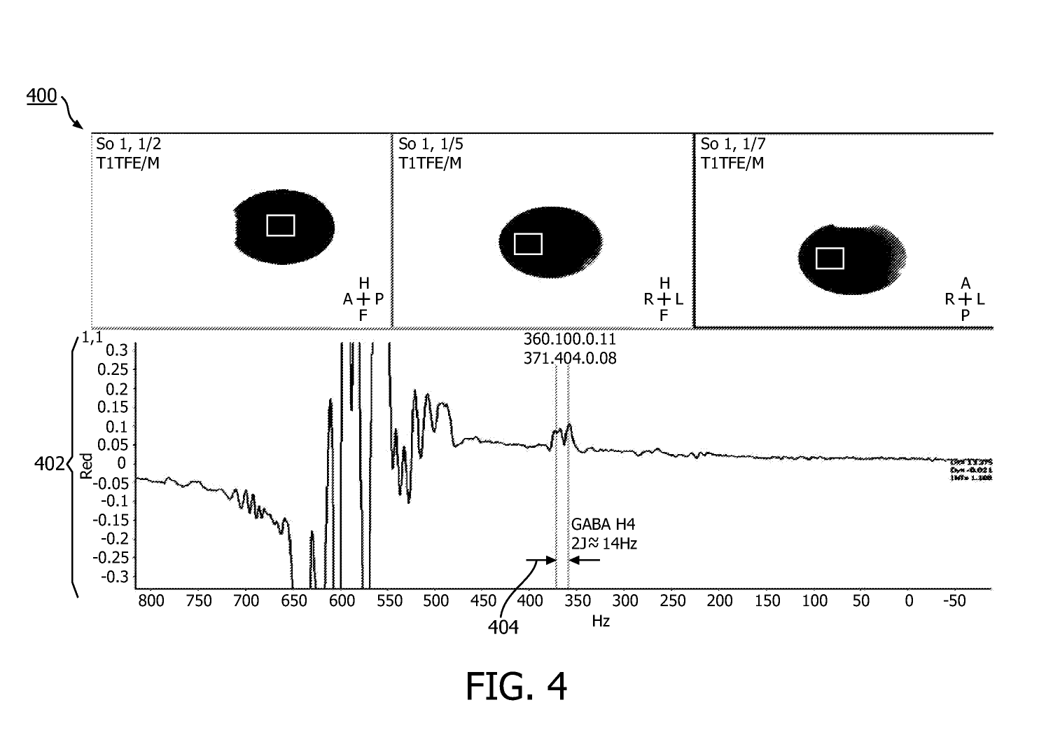

Exemplary Test Results are shown in FIGS. 3 and 4 on Phantoms using an MQF-PRESS pulse sequence in accordance with an embodiment of the present system.

Two exemplary MRI tests were performed using a 3.0T Philips™ Achieva™ system: a first test (Test-1, with the results shown in FIG. 3) was performed using an eight-channel head phase array coil upon a phantom comprising a 10 cm sphere ball filled with solution (containing 20 mM GABA only) to measure MQF efficiency and test stability of the sequence; and a second test (Test-2, with the results shown in FIG. 4) was performed using a thirty-two-channel head phase array coil upon a phantom comprising a 10 cm sphere ball filled with solution (containing 1 mM GABA and 10 mM Cr) to verify suppression of a corresponding Cr signal. In Test-1 and Test-2, a volume of interest (VOI) is 30 mm×30 mm×30 mm, receiver bandwidth is set at 2000 Hz, and a number of data points are illustratively 2048. However in Test-1, the number of repetitions was set to 32 with two dummy scans with water suppression before MQF PRESS sequence; and in Test-2, a number of repetitions was increased to 128 without water suppression before the MQF PRESS sequence.

Exemplary Results of Test-1

FIG. 3 is a graph 300 illustrating spectra of five dynamic scans obtained in accordance with the parameters of Test-1 in accordance with embodiments of the present system. In each of scans (e.g., scan 1 through scan 5), it is seen that the signal amplitude of GABA H4 is consistent in each of the scans. Accordingly, reproducibility of results of the sequence may be considered to be substantially accurate and reproducible. Compared to a regular PRESS sequence at the same TE/TR, the multiple quantum filter (MQF) efficiency of an MQF PRESS sequence performed in accordance with embodiments of the present system is about 40% which is close to that of a stimulated echo acquisition mode (STEAM) sequence which is about 50%. The efficiency in accordance with this embodiment is a ratio of the amplitude of the signal acquired by this sequence and the amplitude of the normal PRESS sequence at the same TR/TE.

As may be readily appreciated, GABA has different spins group marked as H2, H3 and H4, and the signals of these groups show at different respective locations: 2.28 ppm, 1.89 ppm and 3.01 ppm. In the above tests, only the signal at 3.01 ppm is detected which belongs to GABA H4. In accordance with one or more embodiments of the present system, GABA H2, GABA H3 and/or other metabolites may be readily identified.

Exemplary Results of Test-2

FIG. 4 is a screen shot 400 illustrating spectra obtained in accordance with Test-2 parameters upon a 1 mM GABA phantom in accordance with embodiments of the present system. Referring to corresponding spectra in graph 402, signals due to GABA H4 are shown between the arrows 404 with signals due to Cr sufficiently suppressed. The top of the figure shows the location of the voxel in 3-directions from which the signal is detected. The signals, which is left side of the GABA H4 are residual water signals. For in vivo scans of test subjects with low concentrations of GABA such as humans (e.g., human craniums), a thirty-two channel head coil may be preferred as it may provide greater accuracy than may be obtained when using fewer head coils.

Further, although techniques in accordance with embodiments of the present system use phantoms, it should be understood that these techniques may be performed in vivo on test subjects such as humans to determine concentrations of metabolites such as GABA, etc. in a desired VOI such as a volume within a human cranium.

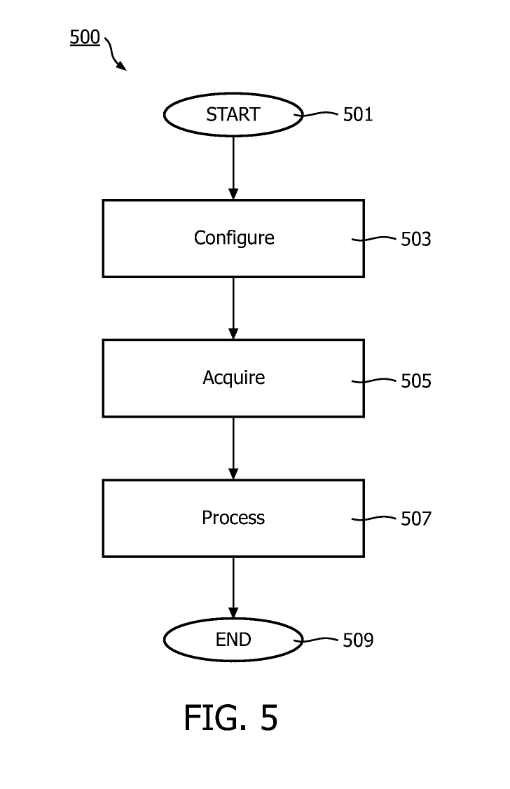

FIG. 5 shows a flow diagram that illustrates an exemplary process 500 performed on an MRI system in accordance with embodiments of the present system. The process 500 may be performed using one or more computers communicating over a network and may obtain information and/or store information using one or more memories which may be local and/or remote from each other. The process 500 may include one of more of the following acts. Further, one or more of these acts may be combined and/or separated into sub-acts, if desired. In operation, the process may start during act 501 and then proceed to act 503.

During act 503, the process may be configured in accordance with selected parameters. Accordingly, the process may obtain from the user (e.g., via a selection of menu items on a user interface such as a GUI, user-entered numerical inputs, etc.) and/or from a memory of the system parameters and/or settings for use with a current test procedure. For example, a user may select a test type (e.g., GABA Cranium Test, etc.) from a plurality of test types presented in a menu of the system or directly entered by a user and may obtain parameters for an associated selected test type (e.g., stored in association with GABA Cranium Test (e.g., see, Table 1, and FIG. 2)) from a memory of the system. Further, settings may be made or adjusted based on the identity of a test subject. However, it is also envisioned that the system may receive selected parameters or settings directly from a user (e.g., a portion of test parameters is shown in Table 1). For example, a user may set a value of BW=60 Hz. The test parameters may include information related to a physical location of the test subject (e.g., in x, y, and/or z axes) during the process 500. After obtaining the selected parameters, the process may configure the MRI system accordingly. During this act, the process may further calculate settings in accordance with the selected parameters and/or known values or settings for corresponding parameters. For example, with reference to Table 1, upon obtaining values for BW, BW_diff and a Flip_Tot, the process may determine PD_tot, N, τ, Flip and θ. Then the process will determine the phases of even pulses in accordance with values obtained for N and θ. Thus, the process may determine values for unknown parameters in accordance with known parameters and a relationship between these parameters as may be set forth in a memory of the system (e.g., see, Table 1). Further, setting information may include timing information for a corresponding TR (e.g., see, FIG. 2). After completing act 503, the process may continue to act 505.

During act 505, the process may acquire MR information (e.g., echo information) from the test subject. Accordingly, the process may control main and/or gradient coils to output desired magnetic fields in accordance with the test parameters (e.g., main magnetic field strength=3T), may control gradient coils to output in accordance with MQF PRESS pulse sequence (e.g., see, FIG. 2)). Further, the process may control the RF transmit antenna(s) to output an RF sequence such as the MQF PRESS pulse sequence for one or more repetition times (e.g., TRs) and may control RF receivers to receive the echo information from the test subject for each repetition time in accordance with embodiments of the present system. After completing act 505, the process may continue to act 507.

During act 507, the process may transform the received echo information into content which may include images, data, and/or graphs (e.g., including metabolite concentration spectra) that can be rendered on, for example, a user interface (UI) of the present system such as on a visual display unit, a speaker, etc. The process may further determine a concentration of a metabolite such as GABA obtained from the FID information and may render this information on a UI of the system for the convenience of a user. After completing act 505, the process may continue to act 507, where it ends.

FIG. 6 is a block diagram of an exemplary MR imaging system 600 in accordance with embodiments of the present system. The imaging system 600 illustratively comprises a set of main magnetic coils 602 for generating a stationary and homogeneous main magnetic field and three sets of gradient coils 603, 604, and 605 for superimposing additional magnetic fields with controllable strength and having a gradient in a selected direction. Conventionally, the direction of the main magnetic field is labeled the z-direction, the two directions orthogonal thereto, and to each other, the x- and y-directions. The gradient coils 603, 604 and 605 are energized via a power supply 611. The imaging device 600 further comprises an RF portion 606 including an RF transmit antenna (Tx) 606T for emitting radio frequency (RF) pulses to a test subject 607 so that the RF pulses are applied to the test subject 607. The RF transmit antenna 606T is coupled to a modulator 609 for generating and modulating the RF pulses to be applied to the test subject 607. Also provided is a receiver 606R for receiving the MR signals, the receiver 606R may be integrated with the transmit antenna 606 (as shown) or, if desired may be separated from it. As may be readily appreciated, the emitted signals are directed to a VOI and the received signals are received from the VOI.

In a case wherein the transmit antenna 606T and receiver 606R are physically the same antenna as shown in FIG. 6, a send-receive switch (SW Tx/Rx) 608 may be arranged to separate the received MR signals (e.g., echoes) from the emitted pulses (RF). The received MR signals may be input to a demodulator 610. The send-receive switch 608, the modulator 609, a demodulator 610, the locator 613, the reconstructor 614, a visual display unit 615, and the power supply 611 for the gradient coils 603, 604, and 605 are controlled by a controller 612 which may control the overall operation of the system 600. Accordingly, the controller 612 controls the phases and amplitudes of the RF signals fed to the RF portion 606 for transmission by the transmit antenna 606T Tx. The controller 612 may include one or more processors such as microprocessors 617 or other logic devices such as microcomputer with a memory and a program control. A memory 618 may store information used by the system such as program information, data, operating parameters, user settings, firmware, user history information, etc. The demodulator 610 is coupled to a reconstructor 614, for example a computer, for transformation of the received signals (e.g., FFT of echoes) into images, data, and/or graphs (e.g., including metabolite concentration spectra) that may be rendered on, for example, a user interface (UI) of the present system such as on the visual display unit 615.

For the practical implementation of the invention, the MR system 600 may include programming for carrying out the above-described acts in accordance with embodiments of the present system. A locator 613 may position the test subject 607 in a desired position and/or orientation relative to one or more of the main magnets coils 602, the gradient coils 603, 604, and 605, and/or the RF portion 606 or parts thereof. The controller 612 may send and/or receive information via any suitable link such as a network 619.

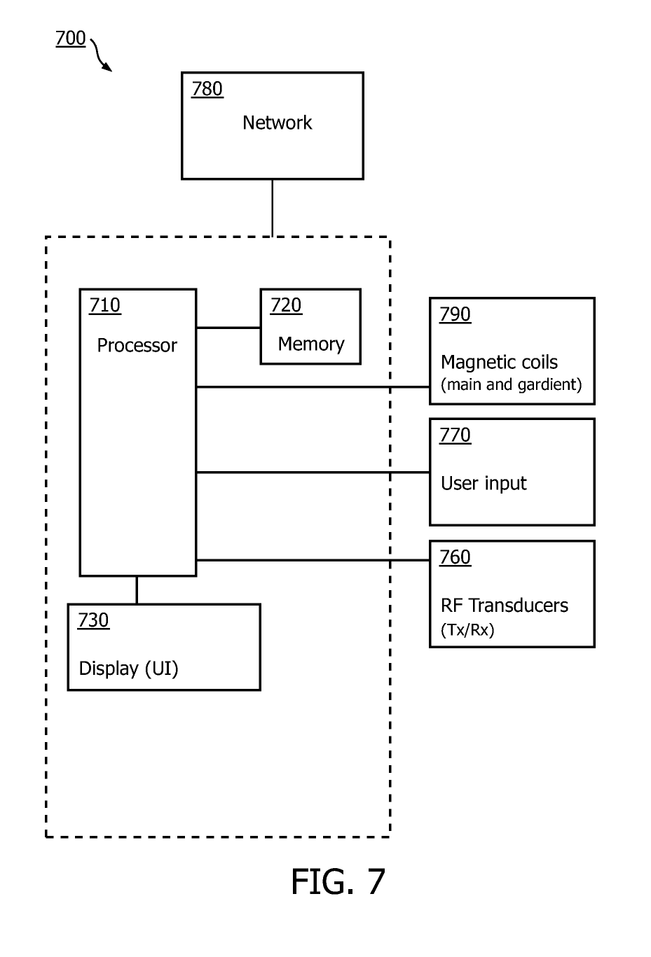

FIG. 7 shows a portion of an exemplary system 700 (e.g., MRI, etc.) in accordance with an embodiment of the present system. For example, a portion of the present system may include a processor 710 operationally coupled to a memory 720, a display 730, RF transducers 760, magnetic coils 790, and a user input device 770. The memory 720 may be any type of device for storing application data as well as other data related to the described operation. The application data and other data are received by the processor 710 for configuring (e.g., programming) the processor 710 to perform operation acts in accordance with the present system. The processor 710 so configured becomes a special purpose machine particularly suited for performing in accordance with embodiments of the present system.

The operation acts may include configured an MRI system by, for example, controlling one or more of a platform, the magnetic coils 790, and/or the RF transducers 760. The platform control a physical location (e.g., in x, y, and z axes) of a test subject and a VOI. The magnetic coils 790 may be controlled to emit a main magnetic field and gradient fields in a desired direction. The controller may control one or more power supplies to provide power to magnetic coils 790 so that a desired magnetic field is emitted at a desired time. The RF transducers 760 may be controlled to transmit RF pulses at the test subject and/or to receive echo information. The echo information may be processed by a reconstructor for transformation of the received signals into content which may include images, data, and/or graphs (e.g., including metabolite concentration spectra) that can be rendered on, for example, a user interface (UI) of the present system such as on the display 730, a speaker, etc. Further, the content may then be stored in a memory of the system such as the memory 720 for later use. Thus, operation acts may include requesting, providing, and/or rendering of content such as, for example, reconstructed image information obtained from the echo information. Further, the processor 710 may determine whether a concentration of a selected metabolite (e.g., GABA) is greater than a threshold value, and if it is determined that the concentration of the selected metabolite is greater than or equal to the threshold value, the processor 710 may take predetermined actions such as by highlighting the concentration of the predetermined metabolite using a red highlight or other suitable color and providing information indicative of the result of the determination to a user (e.g., “GABA levels exceed threshold”) via a UI of the system such as the display 730. However, if it is determined that the concentration of the selected metabolite is less than the threshold value, the processor 710 may take other predetermined actions such as by highlighting the concentration of the predetermined metabolite using a green highlight.

The user input 770 may include a keyboard, a mouse, a trackball, or other device, such as a touch-sensitive display, which may be stand alone or be a part of a system, such as part of a personal computer, a personal digital assistant (PDA), a mobile phone, a monitor, a smart or dumb terminal or other device for communicating with the processor 710 via any operable link. The user input device 770 may be operable for interacting with the processor 710 including enabling interaction within a UI as described herein. Clearly the processor 710, the memory 720, display 730, and/or user input device 770 may all or partly be a portion of a computer system or other device such as MRI device.

The methods of the present system are particularly suited to be carried out by a computer software program, such program containing modules corresponding to one or more of the individual steps or acts described and/or envisioned by the present system. Such program may of course be embodied in a computer-readable medium, such as an integrated chip, a peripheral device or memory, such as the memory 720 or other memory coupled to the processor 710.

The program and/or program portions contained in the memory 720 configure the processor 710 to implement the methods, operational acts, and functions disclosed herein. The memories may be distributed or local and the processor 710, where additional processors may be provided, may also be distributed or may be singular. The memories may be implemented as electrical, magnetic or optical memory, or any combination of these or other types of storage devices. Moreover, the term “memory” should be construed broadly enough to encompass any information able to be read from or written to an address in an addressable space accessible by the processor 710. With this definition, information accessible through a network is still within the memory, for instance, because the processor 710 may retrieve the information from the network for operation in accordance with the present system.

The processor 710 is operable for providing control signals and/or performing operations in response to input signals from the user input device 770 as well as in response to other devices of a network and executing instructions stored in the memory 720. The processor 710 may be an application-specific or general-use integrated circuit(s). Further, the processor 710 may be a dedicated processor for performing in accordance with the present system or may be a general-purpose processor wherein only one of many functions operates for performing in accordance with the present system. The processor 710 may operate utilizing a program portion, multiple program segments, or may be a hardware device utilizing a dedicated or multi-purpose integrated circuit.

Further variations of the present system would readily occur to a person of ordinary skill in the art and are encompassed by the following claims. Through operation of the present system, a virtual environment solicitation is provided to a user to enable simple immersion into a virtual environment and its objects.

Accordingly, it should also be appreciated that embodiments of the present system may detect a first metabolite such as GABA while suppressing a signal spectra due to one or more other metabolites such as CR, MA, N-acytyl aspartate (NAA), choline (Ch), aspartate (Asp), threonine (Thr), glutamate or glutamine (Gix), glutathione (Gsh), myo-inositol (MI), and N-acetyl aspartate (NAA).

Finally, the above-discussion is intended to be merely illustrative of the present system and should not be construed as limiting the appended claims to any particular embodiment or group of embodiments. Thus, while the present system has been described with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

In interpreting the appended claims, it should be understood that:

a) the word “comprising” does not exclude the presence of other elements or acts than those listed in a given claim;

b) the word “a” or “an” preceding an element does not exclude the presence of a plurality of such elements;

c) any reference signs in the claims do not limit their scope;

d) several “means” may be represented by the same item or hardware or software implemented structure or function;

e) any of the disclosed elements may be comprised of hardware portions (e.g., including discrete and integrated electronic circuitry), software portions (e.g., computer programming), and any combination thereof;

f) hardware portions may be comprised of one or both of analog and digital portions;

g) any of the disclosed devices or portions thereof may be combined together or separated into further portions unless specifically stated otherwise;

h) no specific sequence of acts or steps is intended to be required unless specifically indicated; and

i) the term “plurality of” an element includes two or more of the claimed element, and does not imply any particular range of number of elements; that is, a plurality of elements may be as few as two elements, and may include an immeasurable number of elements.

1. A magnetic resonance (MR) system for detecting concentrations of one or more metabolites in a volume of interest (VOI), the system comprising:
at least one controller configured to:
apply to the VOI a multiple quantum filter (MQF) Point Resolved Spetroscopy (PRESS) sequence comprising first and second 90° RF pulses, a third 90° RF pulse, first and second 180° adiabatic pulses, and a composite dual-band delay alternating with nutation for tailored excitation (DANTE) pulse train with a center frequency targeted at Cr and having a plurality of N block pulses, the DANTE pulse train situated in time between the first and second 90° RF pulses, the first and second 180° adiabatic pulses situated in time after the third 90° RF pulse;
detect MR Free Induced Decay (FID) signal emitted from the VOI; and
reconstruct the detected MR FID signal to obtain metabolite spectrum information.

2. The system of claim 1, wherein the first and second 90° RF pulses each have a non-symmetric SINC (Spredrex-type) waveform.

3. The system of claim 1, wherein the third 90° RF pulse has a SINC-type waveform with a 120 Hz excitation bandwidth.

4. The system of claim 1, wherein the first and second 180° adiabatic pulses have an oit_{—}800_{—}6500 pulse-type waveform.

5. The system of claim 1, wherein the DANTE pulse train is defined by two or more of the following parameters:
PD_tot=1.0/BW,
τ=0.5/BW_side,
N=PD_tot/τ,
Flip=2*Flip_tot/N,
θ=−4*τ*BW_diff*180°, and
Phase_even=(N/2−1) θ, (N/2−2) θ, . . . θ, 0, and
Odd_Pulse phase=0
wherein BW refers to an excitation band of an individual band in Hz, PD_tot is a pulse total duration, τ is a block pulse interval, BW_side refers to a sideband of a DANTE pulse, N is an integer corresponding to a total number of block pulses, Flip is a flip angle of each block pulse, Flip_tot refers to a flip angle of the pulse train, θ is phase-shift step of even pulses, BW_diff refers to the distance of dual bands, Phase_even is a phase of even pulses, and Odd_Pulse phase refers to a phase of each odd pulse.

6. The system of claim 1, wherein a selected metabolite of the one of more metabolites is γ-Aminobutyric acid (GABA).

7. The system of claim 6, wherein when the controller is configured to determine a concentration of the selected metabolite in accordance with the metabolite spectrum information.

8. A method of detecting concentrations one or more metabolites in a volume of interest (VOI) using magnetic resonance spectroscopy (MRS), the method performed by a controller of a MRS system and comprising acts of:
applying to the VOI a multiple quantum filter (MQF) Point Resolved Spetroscopy (PRESS) sequence comprising first and second 90° RF pulses, a third 90° RF pulse, first and second 180° adiabatic pulses, and a composite dual-band delay alternating with nutation for tailored excitation (DANTE) pulse train with a center frequency targeted at Cr and having a plurality of N block pulses, the DANTE pulse train situated in time between the first and second 90° RF pulses, the first and second 180° adiabatic pulses situated in time after the third 90° RF pulse;
detecting MR Free Induced Decay (FID) signal emitted from the VOI; and
reconstructing the detected MR FID signal to obtain metabolite spectrum information.

9. The method of claim 8, wherein the first and second 90° RF pulses each have a non-symmetric SINC (Spredrex-type) waveform.

10. The method of claim 8, wherein the third 90° RF pulse has a SINC-type waveform.

11. The method of claim 8, wherein the first and second 180° adiabatic pulses have an oit_{—}800_{—}6500 pulse-type waveform.

12. The method of claim 8, wherein the DANTE pulse train is defined by two or more of the following parameters:
PD_tot=1.0/BW,
τ=0.5/BW_side,
N=PD_tot/τ,
Flip=2*Flip_tot/N,
θ=−4*τ*BW_diff*180°, and
Phase_even=(N/2−1) θ, (N/2−2) θ, . . . θ, 0, and
Odd_Pulse phase=0
wherein BW refers to an excitation band of an individual band in Hz, PD_tot is a pulse total duration, τ is a block pulse interval, BW_side refers to a sideband of a DANTE pulse, N is an integer corresponding to a total number of block pulses, Flip is a flip angle of each block pulse, Flip_tot refers to a flip angle of the pulse, θ is phase-shift step of even pulses, BW_diff refers to the distance of dual bands, Phase_even is a phase of even pulses, and Odd_Pulse phase refers to a phase of each odd pulse.

13. The method of claim 8, wherein a selected metabolite of the one of more metabolites is γ-Aminobutyric acid (GABA).

14. The method of claim 13, further comprising an act of determining a concentration of the selected metabolite in accordance with the metabolite spectrum information.

15. A computer program stored on a computer readable non-transitory memory medium, the computer program configured to detect concentrations one or more metabolites in a volume of interest (VOI) using magnetic resonance spectroscopy (MRS), the computer program comprising:
a program portion configured to:
apply to the VOI a multiple quantum filter (MQF) Point Resolved Spetroscopy (PRESS) sequence comprising first and second 90° RF pulses, a third 90° RF pulse, first and second 180° adiabatic pulses, and a composite dual-band delay alternating with nutation for tailored excitation dual-band delay alternating with nutation for tailored excitation (DANTE) pulse train with a center frequency targeted at Cr and having a plurality of N block pulses, the DANTE pulse train situated in time between the first and second 90° RF pulses, the first and second 180° adiabatic pulses situated in time after the third 90° RF pulse;
detect MR Free Induced Decay (FID) signal emitted from the VOI; and
reconstruct the detected MR FID signal to obtain metabolite spectrum information.

16. The computer program of claim 15, wherein the program portion is further configured to form the first and second 90° RF pulses in accordance with a non-symmetric SINC (Spredrex-type) waveform.

17. The computer program of claim 15, wherein the program portion is further configured to form the third 90° RF pulse in accordance with a SINC waveform.

18. The computer program of claim 15, wherein the program portion is further configured to form the first and second 180° adiabatic pulses in accordance with an oit_{—}800_{—}6500 pulse-type waveform.

19. The computer program of claim 15, wherein the program portion is further configured to form the DANTE pulse train (DBDP) in accordance with two or more of the following parameters:
PD_tot=1.0/BW,
τ=0.5/BW_side,
N=PD_tot/τ,
Flip=2*Flip_tot/N,
θ=−4*τ*BW_diff*180°, and
Phase_even=(N/2−1) θ, (N/2−2) θ, . . . θ, 0, and
Odd_Pulse phase=0
wherein BW refers to an excitation band of an individual band in Hz, PD_tot is a pulse total duration, τ is a block pulse interval, BW_side refers to a sideband of a DANTE pulse, N is an integer corresponding to a total number of block pulses, Flip is a flip angle of each block pulse, Flip_tot refers to a flip angle of the pulse, θ is phase-shift step of even pulses, BW_diff refers to the distance of dual bands, Phase_even is a phase of even pulses, and Odd_Pulse phase refers to a phase of each odd pulse.

20. The computer program of claim 15, wherein the program portion is further configured to determine a concentration of the selected metabolite in accordance with the metabolite spectrum information, and wherein the selected metabolite of the one of more metabolites is γ-Aminobutyric acid (GABA).
